(19) United States
(12) Patent Application Publication
(10) Pub. No.: US 2013/0131571 A1
(43) Pub. Date: May 23, 2013

(54) GAUZE FORMED IN CONTRASTING COLORS

(71) Applicant: Thomas J. Cerra, Carbondale, PA (US)

(72) Inventor: Thomas J. Cerra, Carbondale, PA (US)

(21) Appl. No.: 13/677,250

(22) Filed: Nov. 14, 2012

Related U.S. Application Data
(60) Provisional application No. 61/561,130, filed on Nov. 17, 2011.

(51) Int. Cl. A61F 13/00

(52) U.S. Cl. CPC A61F 13/00059
USPC 602/43

(57) ABSTRACT

A colored gauze scheme helps locate a gauze pad in low light, resulting in cost savings by minimizing lost gauze. Moreover, the colored gauze scheme can provide a method of matching a color to a particular day, allowing medical staff to quickly identify the day the gauze was applied and quickly identify when to change the gauze, thereby reducing the risk of infection.

### CROSS-REFERENCE TO RELATED APPLICATION

This application claims the benefit of priority of U.S. provisional application No. 61/561,130, filed Nov. 17, 2011, the contents of which are herein incorporated by reference.

### BACKGROUND OF THE INVENTION

The present invention relates to gauze pads and, more particularly, to gauze pads formed in a variety of contrasting colors to minimize pad loss in low light as well as to provide a method for bandage changing monitoring and control to help reduce infection.

Presently, gauze pads are formed in a white color. Hospital beds, ambulance gurneys, and most medical bedding are also white. This can result in the easy loss of gauze pads. Moreover, many times, gauze pads need to be changed during the evening or night hours. Medical staff often does this under low light conditions so as not to disturb the patient, or, in a shared room, another patient in the room. White gauze pads on a white sheet are even more prone to loss under low light conditions.

Gauze pads and other bandages need to be changed on a fairly regular schedule to allow proper healing and to help reduce the risk of infection. Medical staff often relies on written charts or other notes to know when the prior gauze was changed and when it should be changed next. This requires accurate documentation and communication to ensure ideal patient care.

As can be seen, there is a need for a methods for distinguishing gauze pads and other medical bandages from bedding as well distinguishing gauze pads based on the date applied.

### SUMMARY OF THE INVENTION

In one aspect of the present invention, a method for visualizing gauze pads comprises obtaining a desired gauze pad, the gauze pad having a non-white color; and applying the non-white color gauze pad to a patient.

In another aspect of the present invention, a method for reducing infection risk in a patient having a would covered by a gauze pad comprises applying a non-white color gauze pad to the patient, the color of the non-white gauze pad corresponding to a weekday; and using the color of the non-white gauze pad to determine a day for properly changing the gauze pad.

These and other features, aspects and advantages of the present invention will become better understood with reference to the following drawings, description and claims.

### BRIEF DESCRIPTION OF THE DRAWINGS

FIG. 1 is a top view of a gauze material with hatching to indicate a blue color, according to an exemplary embodiment of the present invention;

FIG. 2 is a top view of a gauze material with hatching to indicate a green color, according to an exemplary embodiment of the present invention;

FIG. 3 is a top view of a gauze material with hatching to indicate a brown color, according to an exemplary embodiment of the present invention;

FIG. 4 is a top view of a gauze material with hatching to indicate a red/pink color, according to an exemplary embodiment of the present invention;

FIG. 5 is a top view of a gauze material with hatching to indicate a violet/purple color, according to an exemplary embodiment of the present invention; and

FIG. 6 is a top view of a gauze material with hatching to indicate a gray/silver color, according to an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a colored gauze scheme that helps to locate a gauze pad in low light, resulting in cost savings by minimizing lost gauze. Moreover, the colored gauze scheme can provide a method of matching a color to a particular day, allowing medical staff to quickly identify the day the gauze was applied and quickly identify when to change the gauze, thereby reducing the risk of infection.

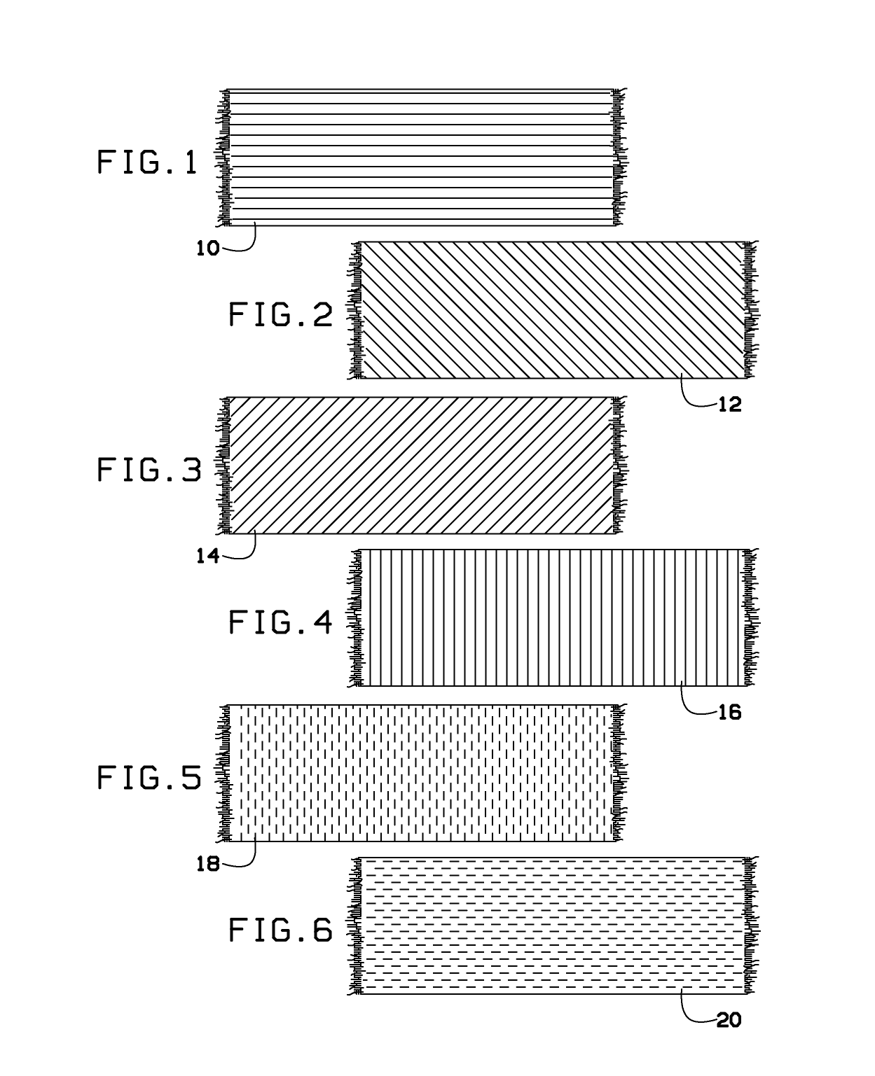

Referring now to FIGS. 1 through 6, gauze 10, 12, 14, 16, 18, 20, can be formed in a variety of colors (non-white gauze), including not only those indicated by the hatching in the Figures, but any one or more distinct colors. By using colored gauze, medical staff can reduce the loss of conventional white gauze in white bedding. Typically, the colored gauze can contrast against the white bedding, resulting in easy visualization of the gauze, even in low light conditions.

The colored gauze can also be used to establish a visual “day of week applied” indicator. A medical facility or other organization could establish guidelines indicating that one color gauze must be used on Monday, another Tuesday, and so forth. In this manner, a medical professional can simply look at the gauze and know that it was applied on Monday and will need changing in another day, for example. In this manner, infection control, due to proper changing of gauze, can be enhanced, improving patient care. In some embodiments, the colored gauze pad can indicate a day in which the gauze should be changed. In either embodiment, the colored gauze pad can help reduce infection by assuring the gauze pad is changed on the proper day of the week.

The colored gauze of the present invention can be formed as gauze pads. The colored gauze pads can be made in conventional gauze pad sizes, such as 2-inches by 2-inches, 4-inches by 4-inches, 6-inches by 8-inches and the like.

The colored gauze of the present invention can be manufactured in various techniques known in the art. Typically, the gauze can be manufactured as gauze pads and stored in closed packaging to keep the gauze sterile until its desired use.

The colored gauze of the present invention can be used by various medical personnel in various departments, including emergency, intensive care, operation, and the like.

It should be understood, of course, that the foregoing relates to exemplary embodiments of the invention and that modifications may be made without departing from the spirit and scope of the invention as set forth in the following claims.

What is claimed is:

1. A method for visualizing gauze pads, comprising:
obtaining a desired gauze pad, the gauze pad having a non-white color; and
applying the non-white color gauze pad to a patient.

2. The method of claim 1, wherein the non-white color gauze pad is formed in a color contrasting from bedding material.

3. The method of claim 1, wherein the non-white color gauze pad contrasts from a color of bedding material to permit visualization in low light conditions.

4. The method of claim 1, wherein the non-white color is selected from a color associated with a weekday in which the gauze pad is applied to the patient.

5. The method of claim 4, wherein the color includes seven colors, one for each weekday.

6. A method for reducing infection risk in a patient having a wound covered by a gauze pad, the method comprising:
applying a non-white color gauze pad to the patient, the color of the non-white gauze pad corresponding to a weekday; and
using the color of the non-white gauze pad to determine a day for properly changing the gauze pad.

7. The method of claim 4, wherein the color includes seven colors, one for each weekday.

8. The method of claim 6, wherein the color indicates one of either a day the gauze pad was applied or a day the gauze pad should be changed.
